(19) United States
(12) Patent Application Publication
(10) Pub. No.: US 2024/0139130 A1
(43) Pub. Date: May 2, 2024

(54) CHEWABLE COMPOSITIONS AND METHODS OF MAKING AND USING THEREOF

(71) Applicant: Trima Israel Pharmaceutical Products Maabarot, Ltd., Kibbutz Ma'abarot (IL)

(72) Inventors: Eitan KADDAR, Kibbutz Ma'abarot (IL); Ran VIGDOR, Ramot HaShavim (IL); Mariana SHWARTZMAN, Nof HaGalil (IL)

(73) Assignee: Trima Israel Pharmaceutical Products Maabarot, Ltd., Kibbutz Ma'abarot (IL)

(21) Appl. No.: 18/567,778

(22) PCT Filed: Jun. 9, 2022
(86) PCT No.: PCT/IL2022/050619

§ 371 Date: Dec. 7, 2023

Related U.S. Application Data
(60) Provisional application No. 63/208,576, filed on Jun. 9, 2021.

(51) Int. Cl. A61K 31/167; A61K 9/00; A61K 47/02; A61K 47/10; A61K 47/12; A61K 47/26; A61K 47/34; A61K 47/36; A61K 47/40

(52) U.S. Cl. CPC A61K 31/167; A61K 9/0056; A61K 47/02; A61K 47/10; A61K 47/12; A61K 47/26; A61K 47/34; A61K 47/36; A61K 47/40

(57) ABSTRACT

A chewable composition is provided. The composition includes a gelling component in a sufficient amount to provide a cohesive gelled product and not more than 1.5% w/w and an active pharmaceutical ingredient (API), wherein the chewable composition has a gel strength up to 10,000 g, measured by pressing force test.

### BACKGROUND OF THE INVENTION

Pharmaceuticals are available in a variety of composition dosages for treating diseases. Dosages that are formulated to be taken orally include tablets, capsules, soft-gels, powders, chewable tablets, and liquid suspensions. Tablets, capsules and soft-gels are dosage forms that can be problematic for individuals that have difficulties swallowing pills such as pediatric and geriatric population. This problem is magnified when the medications need to be taken 2-4 times per day to provide the desired therapeutic effect. Moreover, the need for a source of water or other liquids to assist with swallowing solid composition dosages can complicate administration. Powders are often difficult to administer, and chewable tablets can be hard to chew especially for seniors and young children.

Oral liquid dosage form is the most convenient dosage form for the treatment of pediatric and geriatric population as it can be administered easily. Poor solubility and unpleasant taste are the major issues associated with oral liquid dosage forms. In addition, the dosing with liquid composition dosages is not fixed and needs to be measured on site, which can lead to errors in administration of either too little or too many medications. The unpleasant bitter taste is one of the major problems in completing the treatment in children and in sensitive patients as most of the population cannot tolerate the bitter taste of drugs. The unpleasant taste might lead to poor adherence, which ultimately leads to suboptimal therapeutic value, resulting in ineffective treatment and prolonged illness. Taste is one of the most important parameters governing patient compliance. Undesirable taste is one of several important formulation problems that are encountered with certain drugs. Oral administration of bitter drugs with an acceptable degree of palatability is a key issue for health care providers, especially for pediatric patients. One of the greatest challenges in pediatric pharmacology has been the optimization of oral drug delivery.

Chewable dosage forms are manufactured as solids, such as chewable tablets, or elastic semi-solids such as chewing gums, molded gels, or chewable soft capsules, and make good delivery materials for nutritional such as vitamins and minerals and active pharmaceutical ingredient(s) (API). While elastic semi-solid forms provide better mouth feel and customer acceptance, chewable soft capsules have a further benefit of being totally ingestible and can deliver accurate amounts of active ingredients. Although chewable dosage forms provide an effective dosage system, user acceptance has been limited by their properties as being leathery or rubbery, as well as the difficulties that some users experience in consuming the fractured sheaths after the fills have been released, in terms of texture and mouthfeel. One reason is that many APIs are extremely foul or bitter in taste. The inclusion of the API in a chewable format makes for a foul-tasting product that many would not want. Hence, pharmaceuticals have typically been reserved for non-chewable items that minimize mouth contact.

The problems encountered with currently available formulations and the high ratio of pediatric non-compliance highlight the need for the development of new products that are both easy to administer and capable of providing reliable serum drug concentrations. There remains a need for alternative composition dosages as chewable products, which are low in glycemic index and have palatable taste, for medications to treat various symptoms and illness.

### SUMMARY OF THE INVENTION

In some embodiments, the disclosure relates to a chewable composition, comprising a gelling component in a sufficient amount to provide a cohesive gelled product and not more than 1.5% w/w and an active pharmaceutical ingredient (API), wherein the chewable composition has a gel strength up to 10,000 g, measured by pressing force test. In one embodiment, the composition has a gel strength up to 3,500 g, measured by pressing force test.

In one embodiment, the disclosure relates to a chewable composition comprising sugar alcohols in an amount of not less than 50% w/w.

In one embodiment, the disclosure relates to a chewable composition comprising a low glycemic index sugar component having a glycemic index of not more than 55.

In one embodiment, the disclosure relates to a chewable composition wherein the gelling component does not contain gelatin from animal source.

In one embodiment, the disclosure relates to a chewable composition substantially free of D-glucose, D-fructose, D-sucrose.

In one embodiment, the disclosure relates to a chewable composition, wherein the gelling component comprises carrageenan, pectin or combination thereof. In another embodiment, the gelling component comprises carrageenan. In another embodiment, the gelling component comprises pectin. In another embodiment, the gelling component comprises combination of carrageenan and pectin.

In some embodiments, the disclosure relates to a chewable composition, wherein the active pharmaceutical ingredient (API) comprises antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, an antidepressant, an antipsychotic agent, a cardiovascular agent, an anti-cancer agent, an immune suppressant, a blood thinner, a medication for treating Attention Deficit Hyperactivity Disorder (ADHD), or any combination thereof.

In one embodiment, the active pharmaceutical ingredient (API) comprises a pain or fever reliever agent. In another embodiment, the pain or fever reliever agent comprises acetaminophen.

In another embodiment, the active pharmaceutical ingredient (API) comprises an antibacterial agent. In another embodiment, the antibacterial agent comprises azithromycin.

In some embodiments, the disclosure relates to a chewable composition, wherein the sugar alcohols comprise maltitol, sorbitol liquid, xylitol, erythritol, mannitol, isomalt, lactitol, hydrogenated starch hydrolysates or any combination thereof. In one embodiment, the sugar alcohols comprise maltitol and sorbitol liquid.

In some embodiments, the disclosure relates to the use of the chewable composition as described above, for treating or preventing a disease in a subject in need thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 represents a process flow-manufacturing process for a chewable composition;

FIG. 2 represents the stability results at accelerated conditions of the composition of Table 4;

FIG. 3 represents the dissolution profile at accelerated conditions of the composition of Table 4; and

FIG. 4 represents the comparison of the dissolution profile of three oral dosages comprising acetaminophen.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, the disclosure relates to a chewable composition, comprising a gelling component in a sufficient amount to provide a cohesive gelled product and not more than 1.5% w/w, and an active pharmaceutical ingredient (API), wherein the chewable composition has gel strength up to 10,000 g, measured by pressing force test.

A skilled artisan would understand the meaning of “gel strength” as a measurement of the electrochemical forces within the fluid under static conditions to measure the ability of a colloidal dispersion to develop and retain a gel form. It is the force, expressed in grams, necessary to depress, for example, by 4 mm the surface of a gelatin gel with a standard 0.5″ diameter cylinder probe. The strength is a function of suspended solids, solid contents, temperature, chemical content and time.

In one embodiment, the gel strength is measured by pressing force test. In another embodiment, the gel strength is measured by cutting test.

In one embodiment, the chewable composition has gel strength up to 10,000 g, measured by pressing force test. In another the chewable composition has gel strength up to 9,000 g, measured by pressing force test. In another the chewable composition has gel strength up to 9,500 g, measured by pressing force test. In another the chewable composition has gel strength up to 8,500 g, measured by pressing force test. In another the chewable composition has gel strength up to 8000 g, measured by pressing force test. In another the chewable composition has gel strength up to 7,500 g, measured by pressing force test. In another the chewable composition has gel strength up to 7,000 g, measured by pressing force test. In another the chewable composition has gel strength up to 6,500 g, measured by pressing force test. In another the chewable composition has gel strength up to 6,000 g, measured by pressing force test. In another the chewable composition has gel strength up to 5,500 g, measured by pressing force test. In another the chewable composition has gel strength up to 5,000 g, measured by pressing force test. In another the chewable composition has gel strength up to 4,500 g, measured by pressing force test. In another the chewable composition has gel strength up to 4,000 g, measured by pressing force test. In another the chewable composition has gel strength up to 3,500 g, measured by pressing force test. In another the chewable composition has gel strength up to 3,000 g, measured by pressing force test. In another the chewable composition has gel strength up to 2,500 g, measured by pressing force test. In another the chewable composition has gel strength up to 2,000 g, measured by pressing force test. In another the chewable composition has gel strength up to 1,500 g, measured by pressing force test. In another the chewable composition has gel strength up to 1,000 g, measured by pressing force test.

In one embodiment, the chewable composition has gel strength in the range of 1,000 g-10,000 g, measured by pressing force test. In another embodiment, the chewable composition has gel strength in the range of 1,500 g-9,500 g, measured by pressing force test. In another embodiment, the chewable composition has gel strength in the range of 2,000 g-9,000 g, measured by pressing force test. In another embodiment, the chewable composition has gel strength in the range of 2,500 g-8,500 g, measured by pressing force test. In another embodiment, the chewable composition has gel strength in the range of 3,000 g-8,000 g, measured by pressing force test. In another embodiment, the chewable composition has gel strength in the range of 3,500 g-7,500 g, measured by pressing force test. In another embodiment, the chewable composition has gel strength in the range of 1,500 g-4,000 g, measured by pressing force test. In another embodiment, the chewable composition has gel strength in the range of 1,500 g-3,500 g, measured by pressing force test. In another embodiment, the chewable composition has gel strength in the range of 1,500 g-3,000 g, measured by pressing force test. In another embodiment, the chewable composition has gel strength in the range of 2,000 g-3,000 g, measured by pressing force test.

In one embodiment, the chewable composition has gel strength up to 1,500 g, measured by cutting test. In another embodiment, the chewable composition has gel strength up to 1,400 g, measured by cutting test. In another embodiment, the chewable composition has gel strength up to 1,300 g, measured by cutting test. In another embodiment, the chewable composition has gel strength up to 1,250 g, measured by cutting test. In another embodiment, the chewable composition has gel strength up to 1,200 g, measured by cutting test. In another embodiment, the chewable composition has gel strength up to 1,150 g, measured by cutting test. In another embodiment, the chewable composition has gel strength up to 1,100 g, measured by cutting test. In another embodiment, the chewable composition has gel strength up to 1,050 g, measured by cutting test. In another embodiment, the chewable composition has gel strength up to 1,000 g, measured by cutting test. In another embodiment, the chewable composition has gel strength up to 950 g, measured by cutting test. In another embodiment, the chewable composition has gel strength up to 900 g, measured by cutting test. In another embodiment, the chewable composition has gel strength up to 850 g, measured by cutting test. In another embodiment, the chewable composition has gel strength up to 800 g, measured by cutting test. In another embodiment, the chewable composition has gel strength up to 750 g, measured by cutting test. In another embodiment, the chewable composition has gel strength up to 700 g, measured by cutting test. In another embodiment, the chewable composition has gel strength up to 650 g, measured by cutting test. In another embodiment, the chewable composition has gel strength up to 600 g, measured by cutting test. In another embodiment, the chewable composition has gel strength up to 550 g, measured by cutting test. In another embodiment, the chewable composition has gel strength up to 500 g, measured by cutting test. In another embodiment, the chewable composition has gel strength up to 450 g, measured by cutting test. In another embodiment, the chewable composition has gel strength up to 400 g, measured by cutting test. In another embodiment, the chewable composition has gel strength up to 350 g, measured by cutting test. In another embodiment, the chewable composition has gel strength up to 300 g, measured by cutting test. In another embodiment, the chewable composition has gel strength up to 250 g, measured by cutting test. In another embodiment, the chewable composition has gel strength up to 200 g, measured by cutting test. In another embodiment, the chewable composition has gel strength up to 150 g, measured by cutting test. In another embodiment, the chewable composition has gel strength up to 100 g, measured by cutting test. In another embodiment, the chewable composition has gel strength up to 90 g, measured by cutting test. In another embodiment, the chewable composition has gel strength up to 80 g, measured by cutting test.

In one embodiment, the composition comprises a gelling component, in an amount of no more than 1.5% w/w. In another embodiment, the composition comprises a gelling component, in an amount of 1.5% w/w. In another embodiment, the composition comprises a gelling component, in an amount of 1.4% w/w. In another embodiment, the composition comprises a gelling component, in an amount of 1.3% w/w. In another embodiment, the composition comprises a gelling component, in an amount of 1.2% w/w. In another embodiment, the composition comprises a gelling component, in an amount of 1.1% w/w. In another embodiment, the composition comprises a gelling component, in an amount of 1% w/w. In another embodiment, the composition comprises a gelling component, in an amount of 0.9% w/w. In another embodiment, the composition comprises a gelling component, in an amount of 0.8% w/w. In another embodiment, the composition comprises a gelling component, in an amount of 0.7% w/w. In another embodiment, the composition comprises a gelling component, in an amount of 0.6% w/w. In another embodiment, the composition comprises a gelling component, in an amount of 0.5% w/w. In another embodiment, the composition comprises a gelling component, in an amount of 0.4% w/w.

In one embodiment, the disclosure relates to a chewable composition substantially free of D-glucose, D-fructose or D-sucrose, or any combination thereof. In another embodiment, the chewable composition is substantially free of D-glucose. In another embodiment, the chewable composition is substantially free of D-fructose. In another embodiment, the chewable composition is substantially free of D-sucrose. In another embodiment, the chewable composition is substantially free of a combination of D-glucose, D-fructose and D-sucrose.

A skilled artisan would understand the term “substantially free” to constituent as present in the composition as a contaminant in a trace amount of less than 1% w/w.

In one embodiment, the disclosure relates to a chewable composition comprising sugar alcohols in an amount of not less than 50% w/w. In another embodiment, the disclosure relates to a chewable composition comprising sugar alcohols in an amount of 50% w/w. In another embodiment, the disclosure relates to a chewable composition comprising sugar alcohols in an amount of 55% w/w. In another embodiment, the disclosure relates to a chewable composition comprising sugar alcohols in an amount of 60% w/w. In another embodiment, the disclosure relates to a chewable composition comprising sugar alcohols in an amount of 65% w/w. In another embodiment, the disclosure relates to a chewable composition comprising sugar alcohols in an amount of 70% w/w. In another embodiment, the disclosure relates to a chewable composition comprising sugar alcohols in an amount of 75% w/w. In another embodiment, the disclosure relates to a chewable composition comprising sugar alcohols in an amount of 80% w/w.

In one embodiment, the disclosure relates to a chewable composition, wherein the sugar component has a glycemic index of not more than 55. In one embodiment, the glycemic index is 55. In another embodiment, the glycemic index is 50. In another embodiment, the glycemic index is 45. In another embodiment, the glycemic index is 40. In another embodiment, the glycemic index is 35. In another embodiment, the glycemic index is 30.

In one embodiment, the disclosure relates to a chewable composition, wherein the gelling component comprises agar, carrageenan, processed eucheuma seaweed, locust bean gum, guar gum, tragacanth, acacia gum, xanthan gum, karaya gum, tara gum, konjac, pectin or any combination thereof.

In one embodiment, the disclosure relates to a chewable composition, wherein the gelling component comprises carrageenan, pectin or combination thereof. In another embodiment, the gelling component comprises carrageenan. In another embodiment, the gelling component comprises pectin. In another embodiment, the gelling component comprises combination of carrageenan and pectin.

A skilled artisan would appreciate that carrageenan is a naturally occurring polysaccharide derived from different species of Rhodophyceae (red seaweed). There are three primary families of carrageenan based on the position of sulfate groups and the presence or absence of anhydrogalactose: lambda, iota, and kappa. Carrageenan does not exhibit thermo-reversible gelling property and is stable in accelerated conditions of temperature (e.g., about thirty-seven degrees Celsius (37° C.) to about forty-three degrees Celsius (43° C.)) and relative humidity (about seventy percent relative humidity (70% RH) to about eighty percent relative humidity (80% RH)) in accordance with guidelines set by the International Council for Harmonisation of Technical Requirements for Pharmaceuticals for Human Use (ICH).

In one embodiment, the carrageenan comprises lambda carrageenan, iota carrageenan, kappa carrageenan or combination thereof. In another embodiment, the carrageenan comprises lambda carrageenan. In another embodiment, the carrageenan comprises iota carrageenan. In another embodiment, the carrageenan comprises kappa carrageenan. In another embodiment, the carrageenan comprises lambda carrageenan and iota carrageenan. In another embodiment, the carrageenan comprises lambda carrageenan and kappa carrageenan. In another embodiment, the carrageenan comprises iota carrageenan and kappa carrageenan. In another embodiment, the carrageenan comprises lambda carrageenan, iota carrageenan and kappa carrageenan.

A skilled artisan would appreciate that pectin is a structural acidic heteropolysaccharide contained in the primary and middle lamella and cell walls of terrestrial plants. Its main component is galacturonic acid, a sugar acid derived from galactose. It is produced commercially as a white to light brown powder, mainly extracted from citrus fruits, and is used in food as a gelling agent, particularly in jams and jellies. It is also used in dessert fillings, medicines, sweets, as a stabilizer in fruit juices and milk drinks, and as a source of dietary fiber.

In one embodiment, the disclosure relates to a chewable composition wherein the gelling component does not contain gelatin from animal source.

Active Pharmaceutical Ingredient (API)

In some embodiments, the disclosure relates to a chewable composition, wherein the active pharmaceutical ingredient (API) comprises antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, an antidepressant, an antipsychotic agent, a cardiovascular agent, an anti-cancer agent, an immune suppressant, a blood thinner, a medication for the treatment of Attention Deficit Hyperactivity Disorder (ADHD) or any combination thereof.

A skilled artisan would appreciate the term “active pharmaceutical ingredient (API)” as a substance used in a pharmaceutical dosage form, intended to furnish pharmacological activity or to otherwise have direct effect in the diagnosis, cure, mitigation, treatment, and/or prevention of disease. Active ingredients also include compounds that have, or are thought to have, a direct effect in restoring, correcting, or modifying physiological functions in a patient population (humans or animals).

In one embodiment, the active pharmaceutical ingredient (API) comprises a pain or fever reliever agent. In another embodiment, the pain or fever reliever agent comprises acetaminophen, aspirin, salicylic acid, ibuprofen, naproxen, or any combination thereof. In another embodiment, the active pharmaceutical ingredient (API) comprises acetaminophen.

In one embodiment, the active pharmaceutical ingredient (API) comprises 150 mg acetaminophen in each unit. In one embodiment, the active pharmaceutical ingredient (API) comprises 125 mg acetaminophen in each unit. In one embodiment, the active pharmaceutical ingredient (API) comprises 100 mg acetaminophen in each unit. In one embodiment, the active pharmaceutical ingredient (API) comprises 75 mg acetaminophen in each unit. In one embodiment, the active pharmaceutical ingredient (API) comprises 50 mg acetaminophen in each unit.

In another embodiment, the active pharmaceutical ingredient (API) comprises an antibacterial agent. In another embodiment, the antibacterial agent comprises penicillin, a cephalosporin, a macrolide, a fluoroquinolone, a sulfonamide, a tetracycline, an aminoglycoside, or any combination thereof. In another embodiment, the antibacterial agent comprises bacitracin, clotrimazole, miconazole, penicillin, amoxicillin, cephalexin, erythromycin, clarithromycin, azithromycin, ciprofloxacin, levofloxacin, ofloxacin, co-trimoxazole, trimethoprim, tetracycline, doxycycline, gentamicin, tobramycin, or any combination thereof. In another embodiment, the antibacterial agent comprises azithromycin.

In one embodiment, the active pharmaceutical ingredient (API) comprises 30 mg azithromycin in each unit. In one embodiment, the active pharmaceutical ingredient (API) comprises 50 mg azithromycin in each unit. In one embodiment, the active pharmaceutical ingredient (API) comprises 60 mg azithromycin in each unit. In one embodiment, the active pharmaceutical ingredient (API) comprises 90 mg azithromycin in each unit. In one embodiment, the active pharmaceutical ingredient (API) comprises 100 mg azithromycin in each unit.

In one embodiment, the antidiarrheal agent comprises loperamide, or any combination thereof.

In one embodiment, the antiulcer agent comprises cimetidine, famotidine, nizatidine, ranitidine, misoprostol, sucralfate or any combination thereof.

In one embodiment, the proton pump inhibitor comprises omeprazole, esomeprazole, rabeprazole, pantoprazole, lansoprazole, dexlansoprazole or any combination thereof.

In one embodiment, the laxative or cathartic agent comprises bisacodyl, docusate, miralax, psyllium, senokot, castor oil, magnesium hydroxide, magnesium citrate, magnesium sulfate, lactulose, or any combination thereof.

In one embodiment, the antihistamine comprises cetirizine, diphenhydramine, loratadine, chlorpheniramine, brompheniramine, alimemazine, cyprohetadine, doxyl mine, hydroxyzine, promethazine, or any combination thereof.

In one embodiment, the decongestant comprises pseudoephedrine, oxymetazoline, phenylephrine or any combination thereof. In one embodiment, the antitussive and expectorant comprise guaifenesin, codeine phosphate, dextromethorphan hydrobromide, or any combination thereof.

In one embodiment, the cough suppressant comprises dextromethorphan.

In one embodiment, the medication for treating alcoholism or opioid dependence comprises acamprosate, baclofen, buprenorphine, naloxone, clonidine, di sulfiram, methadone, naltrexone, ondansetron, or any combination thereof.

In one embodiment, the smoke cessation agent comprises nicotine, bupropion, cytosine, varenicline, or any combination thereof.

In one embodiment, the mood stabilizer comprises carbamazepine, gabapentin, lamotrigine, ievetiracetam, lithium salt, oxcarbazepine, topiramate, sodium valproate, divalproex sodium, valproic acid, or any combination thereof.

In one embodiment, the antipsychotic agent comprises aripiprazole, asenapine, chlorpromazine, olanzapine, paliperidone, quetiapine, risperidone, ziprasidone, or any combination thereof.

In one embodiment, the stimulant comprises amphetamine, dexamfetamine, lisdexamfetamine, methylphenidate, dexmethylphenidate, metamfetamine, or any combination thereof.

In one embodiment, the benzodiazepine comprises alprazolam, bromazepam, chlordiazepoxide, clobazam, clonazepam, clorazepate, diazepam, lorazepam, oxazepam, tofisopam, brotizolam, estazolam, flunitrazepam, flurazepam, loprazolam, lormetazepam, midazolam, nimetazepam, nitrazepam, phenazepam, quazepam, temazepam, triazolam, or any combination thereof.

In one embodiment, the anxiolytic agent comprising buspirone, hydroxyzine, meprobamate, pregabalin, or any combination thereof.

In one embodiment, the Z-drug comprises eszopicolone, zaleplon, zolpidem, zopiclone, or any combination thereof.

In one embodiment, the melatonergic agent comprises agomelatine, ramelteon, or any combination thereof.

In one embodiment, the barbiturate comprises amobarbital, secobarbital, butobarbital, cyclobarbital, diazepam, pentobarbital, phenobarbetal, secobarbital, or any combination thereof.

In one embodiment, the antidepressant comprises citalopram, clomipramine, dexopin, escitalopram, fluoxetine, fluvoxarnine, imipramine, mirtazapine, paroxetine, sertraline, trazodone, amitriptyline, doxepin, mianserin, mirtazapine, trazodone, trimipramine, or any combination thereof.

In one embodiment, the antipsychotic agent comprises chlorprothizene, levomepromazine, perazine, promethazine, prothipendyl, sulpiride, thioridazine, zuclopenthixol, perphenazine, beneperidol, bromperidol, fluphenazine, fluspirilen, haloperidoi, pimozide, amisulpride, aripiprazole, asenapine, chloropromazine, clozapine, flupenthixol, lloperidone, melperone, olanzapine, paliperidone, penfluridol, quetiapine, reserpine, risperidone, sertindole, thiothizene, trifluoperazine, zipraisdone, zotepine, pericyazine, carbarn azepine, valproic acid, or any combination thereof.

In one embodiment, the cardiovascular agent comprises an aldosterone receptor antagonist, an angiotensin converting enzyme inhibitor, an angiotensin receptor blocker, a nepriJysin inhibitor, an anti adrenergic agent, an anti anginal agent, an anti arrhythmic agent, an anticholinergic chronotropic agent, an antihypertension agent, an ACE inhibitor, an angiotensin II inhibitor, an anti adrenergic agent, a beta blocker, a diuretic agent, a beta-adrenergic blocker, a calcium channel blocker, a catecholamine, an inotropic agent, a vasodilator, a renin inhibitor, a sclerosing agent, a vasopressin antagonist, a vasopressor, a anti-cholesterol agent, or any combination thereof.

In one embodiment, the antihypertension agent comprises diazoxide, fenoldopam, nitroprusside, a thiazide, or any combination thereof.

In one embodiment, the medication for the treatment of attention deficit hyperactivity disorder (ADHD) comprises methylphenidate, lisdexamfetamine, dexamfetamine, atomoxetine, guanfacine, or any combination thereof.

In some embodiments, the disclosure relates to a chewable composition as described above, wherein the composition comprises 0.1-50% w/w of the API. In one embodiment, the composition comprises 50% w/w of the API. In another embodiment, the composition comprises 45% w/w of the API. In another embodiment, the composition comprises 40% w/w of the API. In another embodiment, the composition comprises 35% w/w of the API. In another embodiment, the composition comprises 30% w/w of the API. In another embodiment, the composition comprises 25% w/w of the API. In another embodiment, the composition comprises 20% w/w of the API. In another embodiment, the composition comprises 15% w/w of the API. In another embodiment, the composition comprises 10% w/w of the API. In another embodiment, the composition comprises 5% w/w of the API. In another embodiment, the composition comprises 1% w/w of the API. In another embodiment, the composition comprises 0.5% w/w of the API. In another embodiment, the composition comprises 0.1% w/w of the API.

Non Active Excipients

In some embodiments, the disclosure relates to a chewable composition as described above, further comprising sweeteners, acids, flavoring agents, taste masking agents, coloring agents, bulking agents, emulsifiers, thickeners, PH adjusters, surfactants, solubility enhancers or any combination thereof.

In one embodiment, the chewable composition may further comprise a sweetener. In one embodiment, the sweetener comprises saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo (monk fruit) sweetener, thaumatin, katemfe fruit extract, siamenoside, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L- -phenylalanine 1-methyl ester, N—[N-[3-(3-hydroxy-4-methoxyphenyl)-3-methylbutyl]-L-.alpha.-aspartyl]-phenylalanine 1-methyl ester, N—[N-[3-(3-methoxy-4-hydroxyphenyl)propyl]-L-.alpha.-aspartyl]-L-phenylal-anine 1-methyl ester, salts thereof or any combinations thereof.

In one embodiment, the chewable composition further comprises an acid component. In one embodiment, the acid component comprises malic acid, fumaric acid, lactic acid, tartaric acid, glucono-delta lactone, salts of gluconic acid, phosphoric acid, succinic acid, adipic acid, acetic acid, citric acid, or any combination thereof.

In one embodiment, the chewable composition further comprises a flavoring agent. In one embodiment, the flavoring agent comprises vanilla, peppermint oil, spearmint oil, eucalyptus oil, cinnamon oil, menthol, monomenthyl succinate, menthol ethylene flycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, or mixtures or derivatives thereof or any combination thereof.

In one embodiment, the chewable composition further comprises a bulking agent. In one embodiment, the bulking agent comprises maltitol syrup, polydextrose, sorbitol, soluble corn fiber, resistant starch, resistant maltodextrin, cellulose, hemicellulose, fructo-oligosaccharides, galacto-oligosaccharides, lactulose, xylo-isomalto-oligosaccharide, soybean oligosaccharide, oligo-glucose, stachyose, lactosucrose, or any combination thereof.

In one embodiment, the chewable composition further comprises a coloring agent. In one embodiment, the coloring agent comprises FD&C Blue No. 1, FD&C Blue No. 2, FD&C Green No. 3, FD&C Red No. 3, FD&C Red No. 40, FD&C Yellow No. 5, FD&C Yellow No. 6, Annatto (E160b), Caramel coloring (E150a-d), Carmine (E120), Dactylopius coccus, Elderberry juice (E163), Lycopene (E160d), Paprika (E160c), Turmeric (E100), Spirulina, Titanium, Dioxide, Iron Oxide Red, Iron Oxide Black, Iron Oxide Yellow, tartrazine (E102), quinoline yellow (E104), sunset yellow (E 110), ponceau (E124), erythrosine (E127), patent blue V (E131), titanium dioxide (E171), aluminum (E173), silver (E174), gold (E175), pigment rubine/lithol rubine BK (E180), calcium carbonate (E170), carbon black (E153), black PN/brilliant black BN (E151), green S/acid brilliant green BS (E142), or any combination thereof. In one embodiment, the chewable composition comprises from about 0.01% to about 1% of coloring agent.

In one embodiment, the chewable composition further comprises a surfactant. In one embodiment, the surfactant comprises anionic surfactant, cationic surfactant, amphoteric surfactant, non-ionic surfactant, or any combination thereof. In another embodiment, the surfactant comprises anionic surfactant. In another embodiment, the surfactant comprises cationic surfactant. In another embodiment, the surfactant comprises amphoteric surfactant. In another embodiment, the surfactant comprises non-ionic surfactant. In one embodiment, the anionic surfactant comprises carboxylate, sulfonate, sulfate groups or any combination thereof. In one embodiment, the cationic surfactant comprises benzalkonium chloride, cetylpyridinium chloride or any combination thereof. In one embodiment, the amphoteric surfactant comprises betaines, sulfobetaine, natural substances such as amino acids and phospholipids or any combination thereof. In one embodiment, the non-ionic surfactant comprises Polyoxyethylene sorbitan fatty acid esters (Polysorbate, Tween®), Polyoxyethylene 15 hydroxy stearate (Macrogol 15 hydroxy stearate, Solutol HS15®), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij®), Polyoxyethylene nonylphenol ether (Nonoxynol®) or any combination thereof. In another embodiment, the surfactant comprises poloxamer.

In one embodiment, the chewable composition further comprises a solubility enhancer. In one embodiment, the solubility enhancer comprises cyclodextrin, hydrochlorthiazide, meloxicam, furosemide, Polyglycolized glyceride, tweens, spans, polyoxyethylene stearates and synthetic block copolymers like poly (propylene oxide)-poly (ethylene oxide)-poly (propylene oxide) like poloxamers based micelles, poly (beta-benzyl-L-aspartate)-b-poly (ethylene oxide), poly (caprolactone)-b-poly (ethylene oxide) or any combination thereof. In another embodiment, the solubility enhancer comprises cyclodextrin.

In one embodiment, the chewable composition further comprises a solvent. In one embodiment, the solvent comprises water, glycerin, propylene glycol, sorbitol liquid, Polyethylene Glycol, Mineral Oil, methyl isobutyl ketoneor or any combination thereof.

In some embodiments, the disclosure relates to a chewable composition as described above, wherein the composition weight is between 1-3.5 grams. In another embodiment, the composition weight is 1 grams. In another embodiment, the composition weight is 1.1 grams. In another embodiment, the composition weight is 1.2 grams. In another embodiment, the composition weight is 1.3 grams. In another embodiment, the composition weight is 1.4 grams. In another embodiment, the composition weight is 1.5 grams. In another embodiment, the composition weight is 1.6 grams. In another embodiment, the composition weight is 1.7 grams. In another embodiment, the composition weight is 1.8 grams. In another embodiment, the composition weight is 1.9 grams. In another embodiment, the composition weight is 2 grams. In another embodiment, the composition weight is 2.1 grams. In another embodiment, the composition weight is 2.2 grams. In another embodiment, the composition weight is 2.3 grams. In another embodiment, the composition weight is 2.4 grams. In another embodiment, the composition weight is 2.5 grams. In another embodiment, the composition weight is 2.6 grams. In another embodiment, the composition weight is 2.7 grams. In another embodiment, the composition weight is 2.8 grams. In another embodiment, the composition weight is 2.9 grams. In another embodiment, the composition weight is 3.0 grams. In another embodiment, the composition weight is 3.1 grams. In another embodiment, the composition weight is 3.2 grams. In another embodiment, the composition weight is 3.3 grams. In another embodiment, the composition weight is 3.4 grams. In another embodiment, the composition weight is 3.5 grams.

In some embodiments, the disclosure relates to a chewable composition as described above, wherein the sugar alcohols comprises maltilol, sorbitol liquid, xylitol, erythritol, mannitol, isomalt, lactitol, hydrogenated starch hydrolysates or any combination thereof.

In one embodiment, the sugar alcohols comprise maltilol. In another embodiment, the sugar alcohols comprise sorbitol liquid. In another embodiment, the sugar alcohols comprise xylitol. In another embodiment, the sugar alcohols comprise erythritol. In another embodiment, the sugar alcohols comprise mannitol. In another embodiment, the sugar alcohols comprise isomalt. In another embodiment, the sugar alcohols comprise lactitol. In another embodiment, the sugar alcohols comprise hydrogenated starch hydrolysates. In another embodiment, the sugar alcohols comprise maltilol and sorbitol liquid.

In some embodiments, the disclosure relates to a chewable composition as described above, wherein the composition has a PH of about 3 to about 7. In one embodiment, the composition has a PH of about 3. In another embodiment, the composition has a PH of about 3.5. In another embodiment, the composition has a PH of about 4. In another embodiment, the composition has a PH of about 4.5. In another embodiment, the composition has a PH of about 5. In another embodiment, the composition has a PH of about 5.5. In another embodiment, the composition has a PH of about 6. In another embodiment, the composition has a PH of about 6.5. In another embodiment, the composition has a PH of about 7.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

In some embodiments, the disclosure relates to a chewable composition as described above, wherein the composition comprises 0%-50% w/w of water. In one embodiment, the composition comprises 50% w/w of water. In another embodiment, the composition comprises 45% w/w of water. In another embodiment, the composition comprises 40% w/w of water. In another embodiment, the composition comprises 35% w/w of water. In another embodiment, the composition comprises 30% w/w of water. In another embodiment, the composition comprises 25% w/w of water. In another embodiment, the composition comprises 20% w/w of water. In another embodiment, the composition comprises 15% w/w of water. In another embodiment, the composition comprises 10% w/w of water. In another embodiment, the composition comprises 5% w/w of water. In another embodiment, the composition comprises 0% w/w of water.

In some embodiments, the disclosure relates to a chewable composition as described above, wherein the composition has Brix value over 80% w/w. In one embodiment, the composition has Brix value of 85% w/w. In another embodiment, the composition has Brix value of 90% w/w. In another embodiment, the composition has Brix value of 95% w/w. In another embodiment, the composition has Brix value of 99% w/w.

A skilled artisan would appreciate that a Brix value, expressed as degrees Brix (° Bx), is the number of grams of sucrose present per 100 grams of liquid. The value represents the strength of the solution as percentage by weight. If the solution contains dissolved solids other than pure sucrose, then the ° Bx only approximates the dissolved solid content.

### Use

In some embodiments, the disclosure relates to the use of the chewable composition as described above, for treating or preventing a disease in a subject in need thereof. In one embodiment, the disclosure relates to the use of the chewable composition as described above, for treating a disease in a subject in need thereof. In another embodiment, the disclosure relates to the use of the chewable composition as described above, for preventing a disease in a subject in need thereof.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

In one embodiment, “preventing” may encompass, inter alia, to delaying the onset of symptoms, preventing relapse to a disease, decreasing the number or frequency of relapse episodes, increasing latency between symptomatic episodes, or a combination thereof.

On one embodiment, the disclosure relates to a composition comprising acetaminophen, for the treatment of pain from headaches, muscle aches, menstrual periods, colds and sore throats, toothaches, backaches, and reactions to vaccinations (shots), fever or any combination thereof.

### EXAMPLES

### Example 1-125 mg Acetaminophen in a 2-Gram Matrix

The purpose of the present example was to test the assay of the content of acetaminophen, of the composition described in Table 1. The assay was performed according to the US pharmacopeia (USP).

TABLE 1
describes the ingredients and their
percentage in the tested composition.

| | Type of | | Percentage |
|---|---|---|---|
| Material | material | Function | per Unit |
| Acetaminophen | Active material | Active material | 6.25% |
| Maltitol | Sugar alcohol | Sweetener | 25% |
| Sorbiltol liquid | Sugar alcohol | Solvent | 25% |
| Glycerin | Lipophilic liquid | Solvent | 3% |
| Propylene glycol | Lipophobic liquid | Solvent | 7% |
| Carrageenan | Polymer | Gelling agent | 1.1% |
| Citric acid | Acid | pH adjustment | 0.06% |
| Masker bitter | Sugar blend | Taste masking | 1% |
| Strawberry flavor | Flavor | Flavor | 1% |
| Water | Solvent | Solvent | q.s |

The assay results of the above composition were 109% w/w of acetaminophen.

In order to improve the assay of acetaminophen and the physical stability of the composition, additional drying time was added to achieve a lower water content and Brix value over 80% w/w, the below composition of Table 2 was tested—

TABLE 2
describes the ingredients and their
percentage in the tested composition.

| | Type of | | Percentage |
|---|---|---|---|
| Material | material | Function | per Unit |
| Acetaminophen | Active material | Active material | 6.25% |
| Maltitol | Sugar alcohol | Sweetener | 25% |
| Sorbiltol liquid | Sugar alcohol | Sweetener | 25% |
| Glycerin | Lipophilic liquid | Solvent | 3% |
| Propylene glycol | Lipophobic liquid | Solvent | 7% |
| Carrageenan | Polymer | Gelling agent | 1.1% |
| Poloxamer | Co-Polymer | Surfactant | 0.3% |
| Citric acid | Acid | pH adjustment | 0.06% |
| Masker bitter | Sugar blend | Taste masking | 1% |
| Strawberry flavor | Flavor | Flavor | 1% |
| Water | Solvent | Solvent | q.s |

The assay results of the above composition were 98.8% w/w of acetaminophen and the Brix value was 88% w/w. In order to improve the physical stability and taste of the composition, the below composition

of Table 3 was tested—

TABLE 3
describes the ingredients and their
percentage in the tested composition.

| | Type of | | Percentage |
|---|---|---|---|
| Material | material | Function | per Unit |
| Acetaminophen | Active material | Active material | 6.8% |
| coated (92%) | | | |
| Maltitol | Sugar alcohol | Sweetener | 35% |
| Sorbiltol liquid | Sugar alcohol | Sweetener | 35% |
| Glycerin | Lipophilic liquid | Solvent | 4% |
| Propylene glycol | Lipophobic liquid | Solvent | 0% |
| Carrageenan | Polymer | Gelling agent | 0.9% |
| Poloxamer | Co-Polymer | Surfactant | 0% |
| Citric acid | Acid | pH adjustment | 0.04% |
| Masker bitter | Sugar blend | Taste masking | 1.2% |
| Strawberry flavor | Flavor | Flavor | 0.3% |
| Water | Solvent | Solvent | q.s |

The assay results of the above composition were 104.1% w/w of acetaminophen.

### Example 2—Manufacturing Process for Chewable Composition

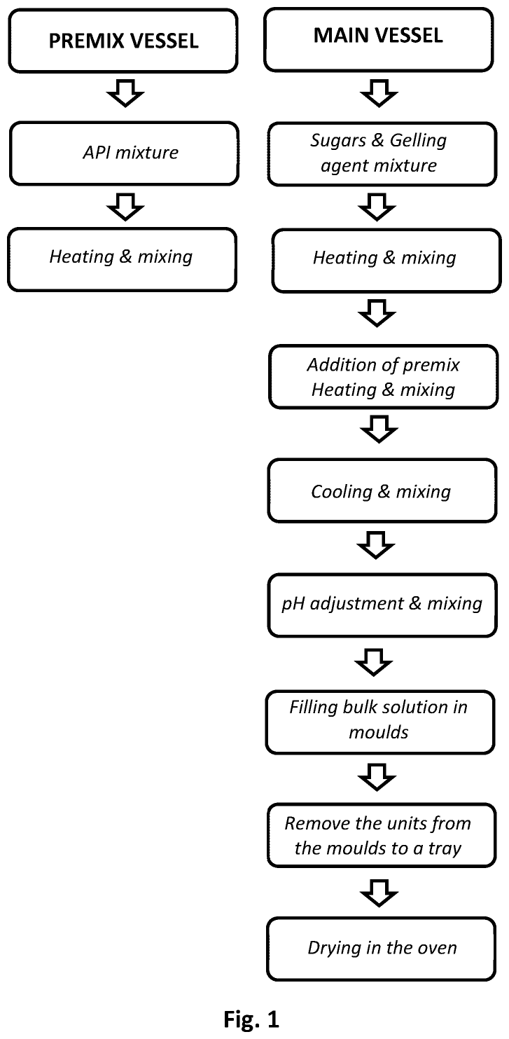

The process for the manufacture of a chewable composition is exemplified in FIG. 1.

The solvent was heated to 80 degrees Celsius at the main vessel. The gelling agent and sugars alcohol were added and mixed to receive homogenous solution. The API with the surfactant were premixed in the solvent. The premix was added to the main vessel and the mixing was continued. The mixture was cooled to 60 degrees Celsius and the flavor and color agent were added with mixing. 10 pH agent was added to adjust the pH with mixing. When the mixture was ready the mixing and heating were continued while filling. The mixture was filled into the molds, the units were removed from the molds to a tray. The tray was transferred to heated oven, for drying for at least 24 hours.

### Example 3—Stability Analysis

The purpose of the present example was to test the stability
of the formulation described in Table 4, below -

| | | |
|---|---|---|
| Material | 100 g | Amount per Batch |

| | | | | |
|---|---|---|---|---|
| Paracetamol | 4.167 | g | 8.333 | g |
| Cyclodextrin | 5 | g | 10 | g |
| Maltitol liquid | 20 | g | 40 | g |
| Sorbitol solution | 30 | g | 60 | g |
| Propylene Glycol | 10 | g | 14 | g |
| Glycerin | 3 | g | 6 | g |
| Titanium dioxide | 0.7 | g | 1.4 | g |
| Citric acid | 0.1 | g | 0.2 | g |
| Trisodium citrate | 0.05 | g | 0.1 | g |
| Masker bitter | 1.7 | g | 3.4 | g |
| Kappa carageenan | 1.2 | g | 2.4 | g |
| Flavor | 0.15 | g | 0.3 | g |
| Colorent | 0.01 | g | 0.02 | g |
| Purified water | 23.923 | g | 53.847 | g |
| | 100 | g | 200 | g |

### Vessel #1—Premix Vessel:

Beta-cyclodextrin and Titanium dioxide were dissolved in a mixture of purified water and propylene glycol at room temperature.

Paracetamol was added to the mixture and the mixture was stirred for 240 minutes.

Masker bitter, citric acid and sodium citrate added to the mixture while mixing.

### Vessel #2—Main Vessel:

Kappa carrageenan was dispersed in a mixture of purified water, sorbitol solution, maltitol liquid and Glycerin at a temperature of 90° C.

The two mixtures were combined in the main vessel at 90° C., strawberry flavor and red color were added while mixing.

The hot bulk was filled into the mold.

After cooling at room temperature, the gel units demolded and transferred to an oven at 40° C. for drying for 20 hours.

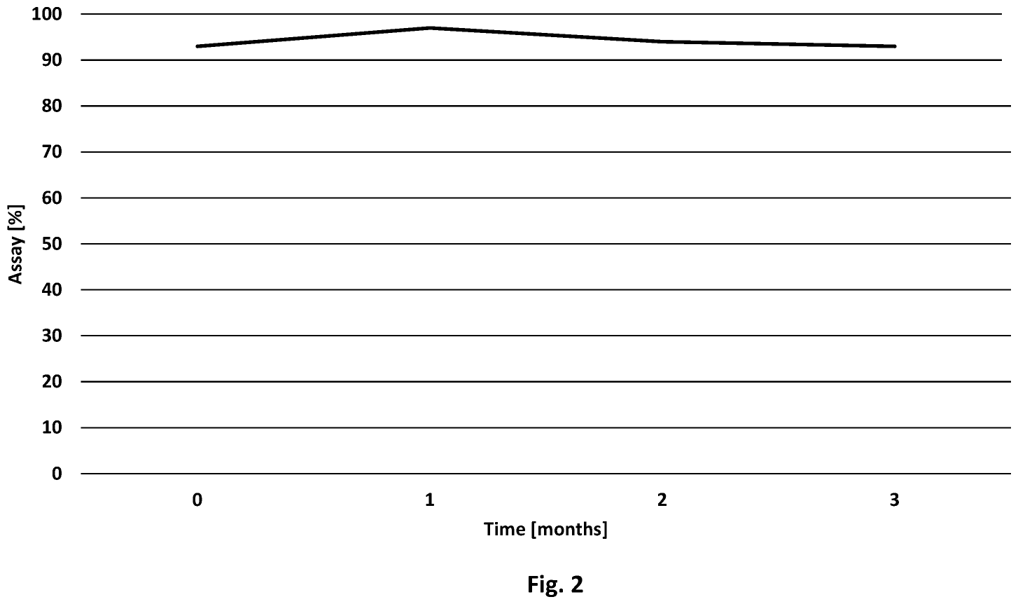

The samples were tested for stability. The samples were placed for three months at accelerated conditions (45° C. & 75% relative humidity (RH)). The stability results are presented in FIG. 2.

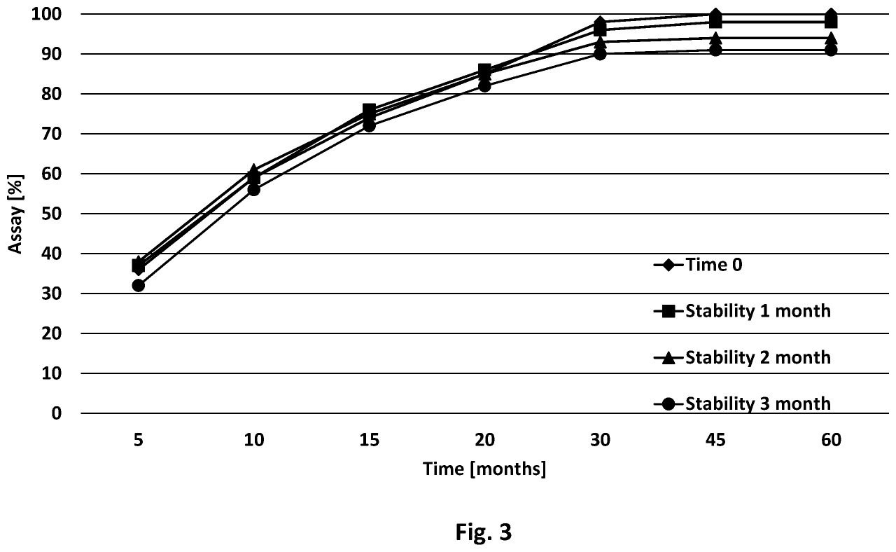

The dissolution profile of the samples was further tested, according to the FDA dissolution method for acetaminophen. The dissolution profile is presented in FIG. 3.

### Example 4—Gel Strength Measuring

The purpose of the present example was to test the gel strength of the chewable composition of the present disclosure (composition described in Table 4), in comparison to gelatin and pectin formulations. The gel strength measurements were performed in two methods, pressing test and cutting test, as described below.

Pressing Test Procedure:
Apparatus P-40 (pressing plate) was assembled into the Stable Micro Systems equipment;
The gel unit was placed on a metal base;
The parameters of speed and depth were adjusted to 2 mm/s & 5 mm;
The test measured the force in grams.

### Cutting Test Procedure:

Apparatus knife (cutting plate) was assembled into the Stable Micro Systems equipment;
The gel unit was placed on a metal base;
The parameters of speed and depth were adjusted to 2 mm/s & 5 mm;
The test measured the force in grams.

The results are presented in Table 5, below -

| Cutting test (g) | Pressing test (g) | |
|---|---|---|
| 750-790 (did not | 2460-3285 | Sample gelatin basis |
| cut through) | | |
| 1070-1100 | <5000 | Sample pectin basis |
| 138-228 | 2634-3329 | E7367/21 |
| 99-135 | 2323-2545 | E7422/22 |
| 189-233 | 1954-2193 | E7394/22 |
| 195-253 | 1252-1304 | E7393/22 |

As can be seen, the chewable compositions of the present disclosure (E7367/21, E7422/22, E7394/22 and E7393/22) present lower gel strength, both in the cutting test and in the pressure test, in comparison with the gelatin and pectin formulation.

### Example 5—Dissolution Comparison

The purpose of the present example was to test the dissolution of three oral compositions, comprising acetaminophen. The oral dosage forms tested were—chewable tablets, oral suspension and solid chewable gel composition according to the present disclosure (composition described in Table 4).

The dissolution assay was performed according to the US pharmacopeia (USP).

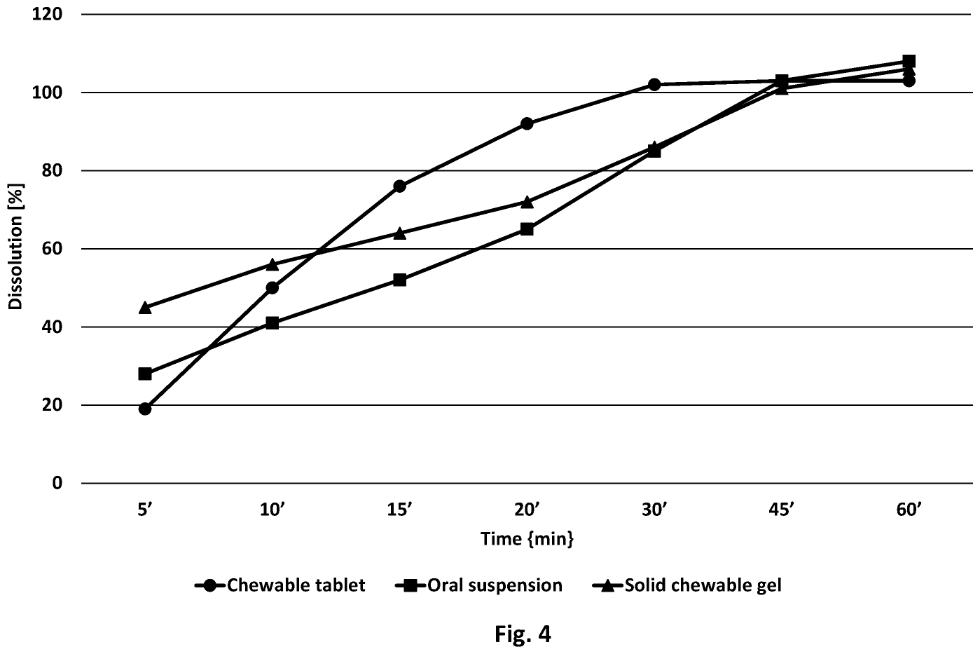

The results are presented in FIG. 4.

### Example 6—Bioavailability Pilot Study

The purpose of the present example is to perform a single dose, comparative bioavailability pilot study of four formulations of acetaminophen 125 mg, under fasting conditions (EMA scope).

Study arms—
Reference product 1—Acetaminophen 500 mg tables.
Reference product 2—Acetaminophen 250 mg/5 ml oral suspension.
Test product—4×125 mg solid chewable gel chewed.
Test product—4×125 mg solid chewable gel swallowed whole.

Periods—4.

Washout—7 days.

Study participants—20 health male and females.

PK samples—0, 0.167, 0.33, 0.5, 0.67, 0.83, 1, 1.25, 1.5, 1.75, 2, 2.5, 3, 4, 6, 8, 10, 12, 16 hours.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A chewable composition, comprising,
a gelling component in a sufficient amount to provide a cohesive gelled product and not more than 1.5% w/w,
an active pharmaceutical ingredient (API),
wherein said chewable composition has a gel strength up to 10,000 g, measured by pressing force test.

2. The chewable composition according to claim 1, wherein said composition has a gel strength up to 3,500 g, measured by pressing force test.

3. The chewable composition according to claim 1, wherein said composition comprises a gelling component in an amount of 1.2% w/w.

4. The chewable composition according to claim 1, wherein said gelling component comprises carrageenan, pectin or combination thereof.

5. The chewable composition according to claim 4, wherein said gelling component comprises carrageenan.

6. The chewable composition according to claim 5, wherein said carrageenan is kappa carrageenan.

7. The chewable composition according to claim 4, wherein said gelling component comprises pectin.

8. The chewable composition according to claim 1, comprising a low glycemic index sugar component having a glycemic index of not more than 55.

9. The chewable composition according to claim 1, wherein said gelling component does not contain gelatin from animal source.

10. The chewable composition according to claim 1, wherein said chewable composition is substantially free of D-glucose, D-fructose, D-sucrose or any combination thereof.

11. The chewable composition according to claim 1, wherein said chewable composition comprises sugar alcohols in an amount of not less than 50% w/w.

12. The chewable composition according to claim 1, wherein said active pharmaceutical ingredient (API) comprises antibacterial agent, an antifungal agent, a pain or fever reliever, an analgesics, an anti-inflammatory agent, a steroid, a diabetic medication, an antidiarrheal agent, an antiulcer agent, a proton pump inhibitor, a laxative or cathartic agent, a diuretic agent, an antacid, an antihistamine, a decongestant, an antitussive or expectorant agent, a cough suppressant, a cold medicine, a motion sickness medication, a medication for treating alcoholism or opioid dependence, a smoke cessation agent, an anti-anxiety agent, an antidepressant, a mood stabilizer, an antipsychotic agent, a stimulant, a benzodiazepine, an anxiolytic agent, a Z-drug, a melatonergic agent, a barbiturate, a antidepressant, an antipsychotic agent, a cardiovascular agent, an anti-cancer agent, an immune suppressant, a blood thinner, or any combination thereof.

13. The chewable composition according to claim 12, wherein said active pharmaceutical ingredient (API) comprises a pain or fever reliever agent.

14. The chewable composition according to claim 13, wherein said pain or fever reliever agent comprises acetaminophen.

15. The chewable composition according to claim 12, wherein said active pharmaceutical ingredient (API) comprises an antibacterial agent.

16. The chewable composition according to claim 15, wherein said antibacterial agent comprises azithromycin.

17. The chewable composition according to claim 1, further comprising sweeteners, acids, flavoring agents, taste masking agents, coloring agents, bulking agents, emulsifiers, thickeners, PH adjusters, surfactants, solubility enhancers, or any combination thereof.

18. The chewable composition according to claim 1, wherein said composition weight is between 1-3.5 grams.

19. (canceled)

20. The chewable composition according to claim 11, wherein said sugar alcohols comprises maltilol, sorbitol liquid, xylitol, erythritol, mannitol, isomalt, lactitol, hydrogenated starch hydrolysates or any combination thereof.

21. (canceled)

22. (canceled)

23. A method of treating or preventing a disease in a subject in need thereof, using a chewable composition according to claim 1.
